(19) United States
(12) Patent Application Publication
(10) Pub. No.: US 2022/0007937 A1
(43) Pub. Date: Jan. 13, 2022

(54) PROBE FOR EYE EXAMINATION USING A DISTAL CONVEX MIRROR

(71) Applicant: JOHNSON & JOHNSON SURGICAL VISION, INC., Santa Ana, CA (US)

(72) Inventors: Assaf Govari, Haifa (IL); Vadim Gliner, Haifa (IL)

(21) Appl. No.: 17/316,959

(22) Filed: May 11, 2021

Related U.S. Application Data
(60) Provisional application No. 63/050,981, filed on Jul. 13, 2020.

(51) Int. Cl. A61B 3/14; A61B 5/00; A61B 1/313; A61B 5/06; A61F 9/007; A61B 1/00

(52) U.S. Cl. CPC A61B 3/14; A61B 5/0079; A61B 1/313; A61B 5/062; A61B 2217/005; A61B 1/00087; A61B 1/00009; A61B 1/00045; A61F 9/00745

(57) ABSTRACT

An ophthalmology apparatus includes a shaft for insertion into an eye, a convex mirror and a camera, and a magnetic orientation sensor. The convex mirror and the camera are coupled at a distal end of the shaft, with the convex mirror located distally to the camera and having an optical path to image onto the camera a region of the eye. The magnetic orientation sensor is coupled to the shaft and configured to measure a roll angle of the shaft.

### CROSS-REFERENCE TO RELATED APPLICATIONS

This application is related to and claims priority to U.S. Provisional Patent Application 63/050,981, filed on Jul. 13, 2020, whose disclosure is incorporated herein by reference.

### FIELD OF THE INVENTION

The present invention relates generally to medical systems, and particularly to phacoemulsification and/or eye examination systems and probes.

### BACKGROUND OF THE INVENTION

Using optical elements in ophthalmic surgical systems, such as in a cataract phacoemulsification system, or in a vitreoretinal surgical system, e.g. vitrectomy (e.g., removal of vitreous humor material), was previously proposed in the patent literature. For example, Chinese Patent Application Publication CN 105640698 describes a femtosecond laser cataract emulsification instrument which comprises an emulsification instrument body. A needle capable of being in contact with a lenticular nucleus of a cataract patient is arranged at the front end of the emulsification instrument body, and a reflector is arranged at the front end of the needle. A splitter is obliquely arranged at the front end in the emulsification instrument body, and a first lens, a rotatable wedge-shaped mirror and a movable second lens are sequentially arranged at the positions, behind the splitter, in the emulsification instrument body. According to the emulsification instrument, full femtosecond lasers are used for tearing bladders, the requirement for the experience of surgeons is low, and the emulsification efficiency is high.

As another example, U.S. Pat. No. 9,364,982 describes an illuminated surgical instrument comprising a cannula and an injection-molded light-sleeve adjacent to and encircling at least a portion of the cannula. The surgical instrument can be a vitrectomy probe having a cutting port disposed at a distal end of the cannula. The light-sleeve can terminate near a distal end of the cannula, for example, near the cutting port of the vitrectomy probe. The light-sleeve is optically coupled to a light source. The light-sleeve can be injection-molded during manufacture using the cannula as an insert for the injection molding. The light-sleeve can be oriented for providing illumination in a direction along a longitudinal axis of the instrument.

### SUMMARY OF THE INVENTION

An embodiment of the present invention that is described hereinafter provides an ophthalmology apparatus including a shaft for insertion into an eye, a convex mirror and a camera, and a magnetic orientation sensor. The convex mirror and the camera are coupled at a distal end of the shaft, with the convex mirror located distally to the camera and having an optical path to image onto the camera a region of the eye. The magnetic orientation sensor is coupled to the shaft and configured to measure a roll angle of the shaft.

In some embodiments, the region of the eye includes a capsular bag.

In some embodiments, the apparatus further includes a phacoemulsification needle coupled at the distal end of the shaft.

In other embodiments, the apparatus further includes a pump configured to aspirate a cataract fragment from the capsular bag via the phacoemulsification needle.

In an embodiment, the apparatus further includes a deflectable distal element coupled at the distal end of the shaft, with the mirror and the camera are coupled to the deflectable distal element, while the magnetic orientation sensor is coupled to the shaft proximally to the deflectable distal element.

In another embodiment, the mirror faces away from the deflectable distal element such that the imaged region of a vitreous humor is in a vicinity of an optic disc region while the deflectable distal element is deflected away from the optic disc.

In some embodiments, the apparatus further includes a display, and a processor. The processor is configured to (a) receive respective signals from the magnetic orientation sensor while the shaft is rotated to respective orientations, (b) compute respective roll angles of the shaft at the respective orientations responsively to the respective received signals, (c) output a notification responsively to at least one of the computed roll angles, and (d) render to the display respective images captured by the camera of respective inner regions of the eye.

In some embodiments, the processor is further configured to determine when one of the computed roll angles exceeds a predefined limit, and output a notification indicating that the one of the computed role angles exceeds the predefined limit.

In other embodiments, the apparatus further includes a deflectable distal element coupled at the distal end of the shaft, with the mirror and the camera coupled to the deflectable distal element, while the magnetic orientation sensor is coupled to the shaft proximally to the deflectable distal element, and the processer is further configured to (i) compute a deflection of the deflectable distal element, and (ii) render to the display an indication of the computed deflection.

In an embodiment, the apparatus further includes a puller wire passing via a lumen of the shaft, a distal end of the puller wire being connected to the deflectable distal element so that pulling a proximal end of the puller wire deflects the deflectable distal element. The processor is configured to compute the deflection of the deflectable distal element responsively to an electrical impedance of at least part of the puller wire.

In another embodiment, the processor is configured to (a) render to the display the respective images captured by the camera of the respective inner regions of the eye on a three-dimensional surface responsively to the computed deflection and the computed roll angles, (b) receive a user interface command to rotate the three-dimensional surface, and (c) render to the display a rotated view of the three-dimensional surface responsively to the received user interface command.

There is additionally provided, in accordance with another embodiment of the present invention, an ophthalmology method, including inserting into an eye a handpiece including a shaft, a convex mirror, a camera, and a magnetic orientation sensor, wherein the convex mirror and the camera are coupled at a distal end of the shaft, wherein the convex mirror is located distally to the camera and includes an optical path to image onto the camera a region of the eye, and wherein the magnetic orientation sensor is coupled to the shaft and configured to measure a roll angle of the shaft. Respective signals are received from the magnetic orientation sensor while the shaft is rotated to respective orientations. Respective roll angles of the shaft at the respective orientations are computed responsively to the respective received signals. A notification is outputted responsively to at least one of the computed roll angles. Respective images are rendered to a display, which are captured by the camera of respective inner regions of the eye.

The present invention will be more fully understood from the following detailed description of the embodiments thereof, taken together with the drawings in which:

### BRIEF DESCRIPTION OF THE DRAWINGS

FIG. 1 is a schematic, pictorial view, along with an orthographic side view and a cross sectional view, of a phacoemulsification apparatus comprising a probe for phacoemulsification and eye examination, in accordance with an embodiment of the present invention;

FIG. 2 is a schematic, orthographic side view of an eye examination probe comprising a deflectable distal element for use in vitrectomy, in accordance with an embodiment of the present invention;

FIG. 3 is a cross-sectional view of the probe of FIG. 2, in accordance with an embodiment of the present invention;

FIG. 4 is a schematic pictorial view of a distal end of the probe of FIG. 2 being used in an examination procedure to inspect a vitreous humor region in the vicinity of an optical disc, in accordance with an embodiment of the present invention; and

FIG. 5 is a flow chart schematically illustrating a method for using the probe of FIG. 2 to examine an eye after vitrectomy, in accordance with an embodiment of the present invention.

### DETAILED DESCRIPTION OF EMBODIMENTS

### Overview

Eye surgeries may cause complications that may require specialty eye examination tools, e.g., to manage a corrective action, such as described in embodiments of the present invention below.

For example, while cataract surgery is usually performed without complications, in some eyes, fragments of the cataract may fall into the back of the capsular bag. When cataract pieces or lens fragments remain in the eye after surgery, a severe inflammatory reaction can occur. As another example, in vitreoretinal surgery the vitreous humor may require inspection to ensure no complications developed (such as protein deposits in vicinity of the retina).

An apparatus that addresses the above challenges using a distal camera to look, for example, for cataract fragments is described in U.S. patent application Ser. No. 16/704,054, filed Dec. 5, 2019, titled “eye examination apparatus,” which is assigned to the assignee of the present patent application, and whose disclosure is incorporated herein by reference.

Embodiments of the present invention that are described hereinafter provide an ophthalmology apparatus having improved eye examination capabilities that, for example, reduce post-operative complications. Some disclosed embodiments provide an eye examination apparatus that includes a probe, which is inserted into the eye after cataract removal has been performed, to search for cataract pieces or lens fragments after cataract surgery. Different probes are provided for different clinical needs, that include a miniature (e.g., no larger than a 1 mm size cube) distal convex mirror which images a view of either the capsular bag, using one type of probe, or the vitreous humor, using another type of probe, onto a camera located proximally to the mirror. The convex mirror is generally placed close to the distal edge of the probe, for example within 5 mm of the distal edge.

In some embodiments, a convex parabolic mirror is used, which has advantageous optical properties of a sharp focus due to lack of spherical aberrations. In other embodiments, a spherical mirror is sufficient, for example by using corrective optics between the mirror and the camera and/or an algorithm for aberration correction.

In some embodiments the optical path between the mirror and the camera passes inside the probe. In other embodiments, the mirror and the camera protrude from the probe, and the optical path between them is external to the probe.

The probe further includes a magnetic orientation sensor which provides signals that the processor uses to compute a roll angle of the probe and thereby (i) label each view with an orientation from which the view was taken, and (ii) determine whether the probe has been rotated completely, e.g., has exceeded a predefined limit, such as 360°, inside the eye. The processor may then output a notification (e.g., visual, audible and/or tactile) to indicate complete rotation. The processor may further output a notification responsively to one or more of the computed roll angle values. For example, the computed roll angle values may be rendered to a display along with respective images captured by the camera.

In some embodiments, the eye examination probe is implemented as part of a phacoemulsification probe including a needle coupled to a shaft of the probe at its distal end, where the convex mirror faces away from a shaft of the probe. When cataract pieces or lens fragments are found in the camera images, a pump may be used to aspirate them from the capsular bag.

In some embodiments, the eye examination probe is implemented separately from the phacoemulsification probe. Such a probe may include, at its distal end, a deflectable distal element to which the mirror and the camera are coupled. In some embodiments, this element may be deflected over a range of at least 120° from an axis of the section of the shaft proximal to the deflectable distal element. Imaging the interior of the eye using a mirror in this way enables the deflection of the distal edge of the probe away from sensitive regions of the capsular bag, such as its posterior portion.

The shaft of the deflectable eye-examination probe may include a puller wire passing within a lumen, where the distal end of the puller wire is connected to the deflectable distal element, such that pulling the proximal end of the puller wire in a proximal direction deflects the distal element. Deflecting this element allows the physician to adjust the mirror toward different portions of the interior of the eye. A processor computes this deflection using any suitable method, for example responsively to an increased impedance of at least part of the puller wire as it is stretched.

In embodiments where the probe includes the deflectable distal element, the processor may render an indication of the computed deflection on the display.

In some embodiments, the captured images may be rendered to a three-dimensional (3D) surface, such as a sphere, according to the computed roll angle values and deflections at which the respective captured images were captured. The 3D surface may be then manipulated by a user to see different sides of the surface.

### Cataract Surgery and Capsular Bag Inspection System

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which may include an ultrasonic handpiece with a needle. The tip of the needle vibrates at ultrasonic frequency to sculpt and emulsify the cataract while a pump aspirates particles and fluid from the eye through the tip. An alternative solution includes using a pulsed laser to emulsify the cataract. Either way, aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule restoring the patient's vision.

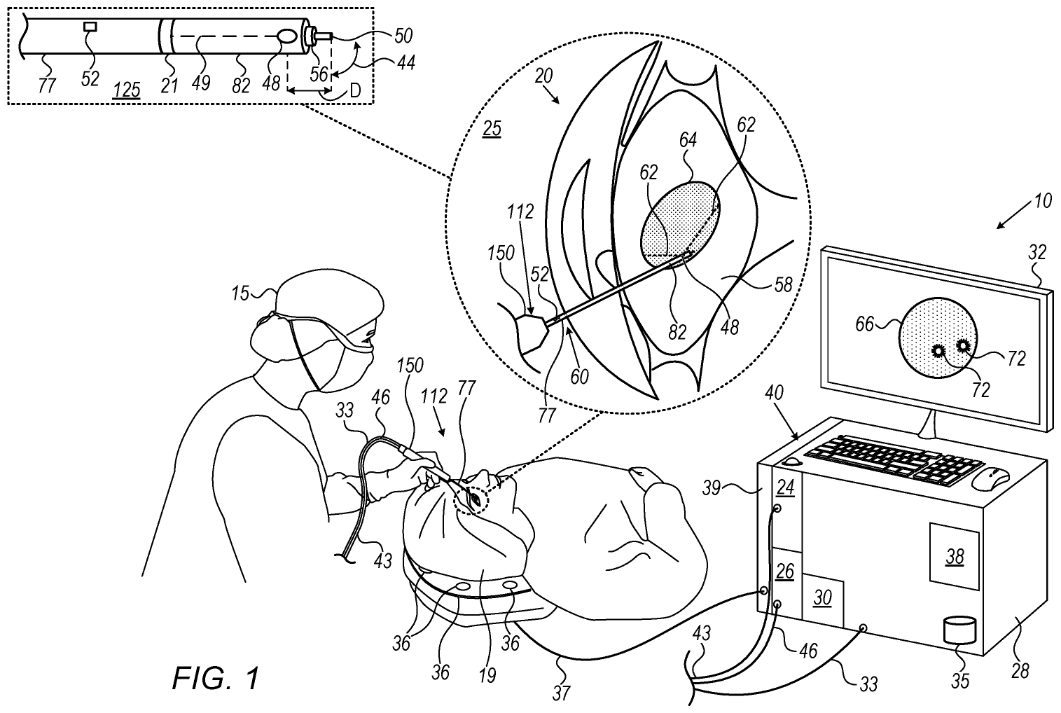

FIG. 1 is a schematic, pictorial view, along with a cross sectional view 25 and an orthographic side view 125, of a phacoemulsification apparatus comprising a probe 112 for phacoemulsification and eye examination, in accordance with an embodiment of the present invention.

As seen in the figure and in inset 25, during an eye examination procedure, a physician 15 inserts a distal end 82 of a shaft 77 of probe 112 into a capsular bag 58 of an eye 20 of a patient 19 via an incision 60 made during the cataract surgery.

As seen in inset 125, a convex mirror 48 is coupled at a distal portion of distal end 82, and a camera 21 is located at a proximal portion of distal end 82, with an optical path 49 (e.g., a lumen or an optical guide) in between. A needle 50 is coupled at the distal edge of distal end 82, and a magnetic orientation sensor 52 coupled at a proximal section of shaft 77. Needle 50 is hollow and its lumen is used as an aspiration channel. Probe 112 also includes an irrigation sleeve 56 around needle 50.

Convex mirror 48 faces away from distal end 82 so that the field of view of mirror 48 faces away from the distal end 82. In some embodiments, an axis of the field-of-view is generally perpendicular to a longitudinal axis of distal end 82. The field-of-view of mirror 48 may nevertheless face any suitable direction as long as an optical path to camera 21 enables mirror 48 to image an outside object onto camera 21.

The field-of-view of mirror 48 is shown in inset 25 by way of lines 62 and an image footprint 64. An image 66 including cataract fragments 72, imaged by convex mirror 48 and captured by a camera 21, is conveyed via cable 33 as electrical signals to processor 38 that displays the image 66 on a display 32. In other embodiments, probe 112 is connected wirelessly to a console 28 to convey electrical signals between probe 112 and the console.

In some embodiments, mirror 48 is coupled within approximately D=5 mm of the distal edge of needle 50.

Phacoemulsification probe 112 includes other items (not shown), such as a piezoelectric crystal coupled to a horn to drive the needle 50 to vibrate at a trajectory 44.

The piezoelectric crystal is configured to vibrate needle 50 in a resonant vibration mode. The vibration of needle 50 is used to break a cataract into small pieces during a phacoemulsification procedure.

In the shown embodiment, console 28 comprises a piezoelectric drive module 30, coupled with the piezoelectric actuator using electrical wiring running in cable 33. Drive module 30 is controlled by a processor 38 and conveys processor-controlled driving signals via cable 33 to maintain needle 50 at maximal vibration amplitude of trajectory 44. The drive module may be realized in hardware or software, for example, in a proportional-integral-derivative (PID) control architecture.

As noted above, to track a roll angle of shaft 77, magnetic orientation sensor 52 provides orientation-indicative signals when its orientation is changed with respect to a magnetic field generated by radiators 36 (i.e., orientation relative to the magnetic tracking frame of reference). In some embodiments, the magnetic orientation sensor 52 includes a single axis coil which provides the orientation signals which are conveyed over cable 33. Driving signals to magnetic field radiators 36 are conveyed over cable 37 from a magnetic tracking module 39 to a location pad positioned beneath, or around, the patient's head. Module 39 is controlled by a processor 38, which is also provided with the sensed signals.

The operation of a magnetic tracking system and its use in the context of probe-based procedures are described in U.S. Patent Application Publication 2014/0024969, which is assigned to the assignee of the present patent application, which document is incorporated by reference.

In some embodiments, for the tracking of orientation to be effective, frames of reference of a CT (computerized tomography) image of patient 19 are registered with the magnetic tracking system. While the CT image may typically comprise a magnetic resonance imaging (MRI) image or a fluoroscopic image, the image in the description herein is assumed to comprise, by way of example, a fluoroscopic CT image.

In the shown embodiment, during the phacoemulsification procedure, a pumping sub-system 24 comprised in a console 28 pumps irrigation fluid from an irrigation reservoir to irrigate the eye via needle 50. In other embodiments, gravity fed irrigation may be used without use of a pump. The fluid is initially pumped via a tubing line 43 running from console 28 to probe 112. Eye fluid and waste matter (e.g., emulsified parts of the cataract) are aspirated via needle 50 to a collection receptacle by a pumping sub-system 26, also comprised in console 28, using tubing line 46 running from probe 112 to console 28.

Processor 38 may receive user-based commands via a user interface 40, which may include stroke amplitude settings of needle 50, and turning on irrigation and/or aspiration. For example, using a foot pedal (not shown) in one position, only irrigation is activated, and in foot pedal position two, irrigation and aspiration are both activated. In foot pedal position three, vibration of needle 50 is added. In an embodiment, the functions of user interface 40 and display 32 may be combined by using a touch screen graphical user interface. Additionally, or alternatively, processor 38 may receive user-based commands from controls located in handle 150, to, for example, select a trajectory for needle 50 or to apply irrigation and/or aspiration.

Some or all of the functions of processor 38 may be combined in a single physical component or, alternatively, implemented using multiple physical components. These physical components may comprise hard-wired or programmable devices, or a combination of the two. In some embodiments, at least some of the functions of processor 38 may be carried out by suitable software stored in a memory 35. This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 may include further elements, which are omitted for clarity of presentation. For example, physician 15 typically performs the procedures using a stereo microscope or magnifying glasses, neither of which are shown. Physician 15 may use other surgical or invasive diagnostic tools in addition to probes 112, which are also not shown in order to maintain clarity and simplicity of presentation.

### Eye Examination Probe Having a Distal Convex Mirror for Use In Vitreoretinal Surgery

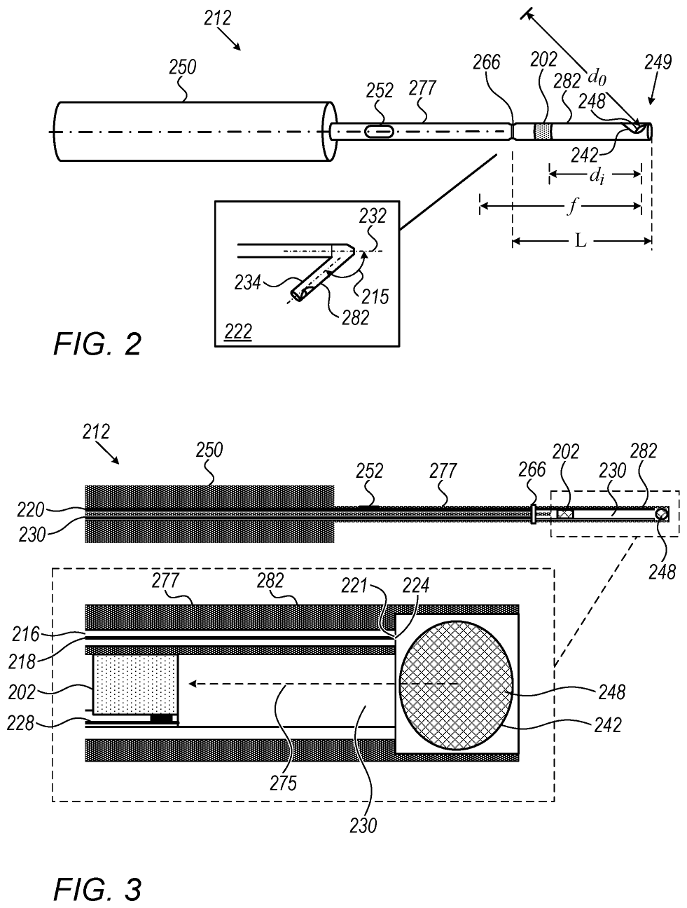

FIG. 2 is a schematic, orthographic side view of an eye examination probe 212 comprising a deflectable distal element 282, for use in vitreoretinal surgery, e.g. vitrectomy, in accordance with an embodiment of the present invention. While the following description is considering use of probe 212 in the vitreous humor, probe 212 may be also used in capsular bag inspection (e.g., during or after cataract surgery).

As seen in FIG. 2, probe 212 includes a handle 250 and a shaft 277 which is configured for insertion into a vitreous humor of an eye. As further seen, a magnetic orientation sensor 252 is coupled in shaft 277, proximally to deflectable distal element 282.

Shaft 277 includes a deflectable distal element 282 having a length L at a distal end of the shaft. Deflectable distal element 282 is normally straight, using, for example, a self-opening hinge 266, and a puller wire (shown in FIG. 3) to deflect the element. Deflectable distal element 282 is coupled with a convex mirror 248 having a focal length f. The center of the field-of-view of mirror 248 is generally at an angle (e.g., 45°) to a longitudinal axis of the deflectable distal element 282. A maximal possible field-of-view of mirror 248 may be determined by the measure of aperture 242 in element 282.

Convex mirror 248 may be coupled with deflectable distal end 282 with a transparent or translucent window between mirror 248 and an optical path to a camera 202 (such as optical path 275 seen in FIG. 3). Alternatively, mirror 248 may be embedded in a transparent case 249 made of a plastic material having a sufficiently low refractive index (i.e., ˜1.4) to match that of the vitreous humor (i.e., ˜1.34). Antireflection coatings may be used as necessary.

Camera 202, disposed in deflectable distal element 282, is located at distance dᵢ<f, L proximally to spherical convex mirror 248, where dᵢ is the imaging plane of spherical convex mirror 248. An optical path (seen in FIG. 3 as optical path 275) exists between mirror 248 and camera 202. Length d₀ is the distance between the mirror to an imaged object (e.g., a material 172 in the vitreous humor), and is given by:

### Eq. 1

$$\frac{1}{{d}_{o}} = \frac{1}{{d}_{i}} - \frac{1}{f}$$

Proper selection of focal length f and dᵢ takes into account the practical range of distances d₀ that are required by the clinical application and are achievable by moving shaft 277 inside the vitreous humor.

In some embodiments, camera 202 fits into a space of about a 1 mm cube or less. Any suitable camera may be used for camera 202. For example, the OVM6948 camera, commercially available from of Omnivision® of Santa Clara, Calif., USA, has a package dimensions of 650×650×1158 micrometers.

Deflectable distal element 282 may have any suitable outside diameter, typically 1-2 mm. Shaft 277 may have any suitable outside diameter, for example, 2-4 mm. Deflectable distal element 282 may have any suitable length L, and in some embodiments, a length L of less than 5 mm.

Deflectable distal element 282 may deflect by any suitable angle. In some embodiments, one of which is seen in inset 222, a longitudinal axis 234 of the deflectable distal element 282 is configured to deflect by an angle 215 of at least 120° from longitudinal axis 232 of a section of shaft 277 proximal to the deflectable distal element 282. Handle 250 includes controls for a deflection angle of deflectable distal element 282, among other functions.

FIG. 3 is a cross-sectional view of the probe of FIG. 2, in accordance with an embodiment of the present invention. As seen, an optical path 275, formed as a lumen 230 or an optical guide, allows mirror 248 to image an object outside element 282 onto camera 202.

Note that, using a convex mirror, any outside object imaged onto the camera plane is smaller than its actual size, so even a field-of-view as large as several millimeters or more can be reduced by proper selection of a magnification factor using Eq. 1.

In another embodiment (not shown), the mirror and the camera protrude outside of deflectable distal element 282, providing an uninterrupted optical path between them.

FIG. 3 further shows that shaft 277 includes a lumen 216 and a puller wire 218 passing in a lumen 116 of shaft 277, the puller wire having a proximal end 220 and a distal end 221. Distal end 221 of puller wire 218 is connected to a distal portion 224 of the deflectable distal element 282 so that pulling the proximal end 220 of puller wire 218 in a proximal direction generates a deflection in the deflectable distal element 282 as shown in inset 222 of FIG. 2. Puller wire 218 may be implemented using any suitable material, for example, stainless steel or nickel-titanium alloy. Deflectable distal element 282 may be configured as deflecting in one direction only, or in two directions by using more than one puller wire. Deflectable distal element 282 may be implemented using any suitable flexible biocompatible material such as polyimide or polyurethane. Deflectable distal element 282 may be resilient. Shaft 277 may be implemented using any suitable biocompatible material, for example, polyimide. Lumen 230 in shaft 277 contains electrical wires 228 to connect camera 202 to processor 20.

The angle of deflection between axis 234 of deflectable distal element 282 and axis 232 of shaft 277 may be computed based on calibrating the angle of deflection as a function of the stretching of puller wire 218. In some embodiments, one electrical wire may be connected to the distal end 221 of puller wire 218, and another to the proximal end 220 of puller wire 218, so that an impedance of the puller wire 218 may be measured, for example, by processor 20, in order to determine angle of deflection of the deflectable distal element 282 as a function of impedance in puller wire 218. A controller on handle 250 may be used to adjust puller wire 218, which in turn adjusts the deflection. In some embodiments, a calibrated scale may be added to the controller or to the handle 250 showing the different angles of deflection that may be caused by certain adjustments of the controller.

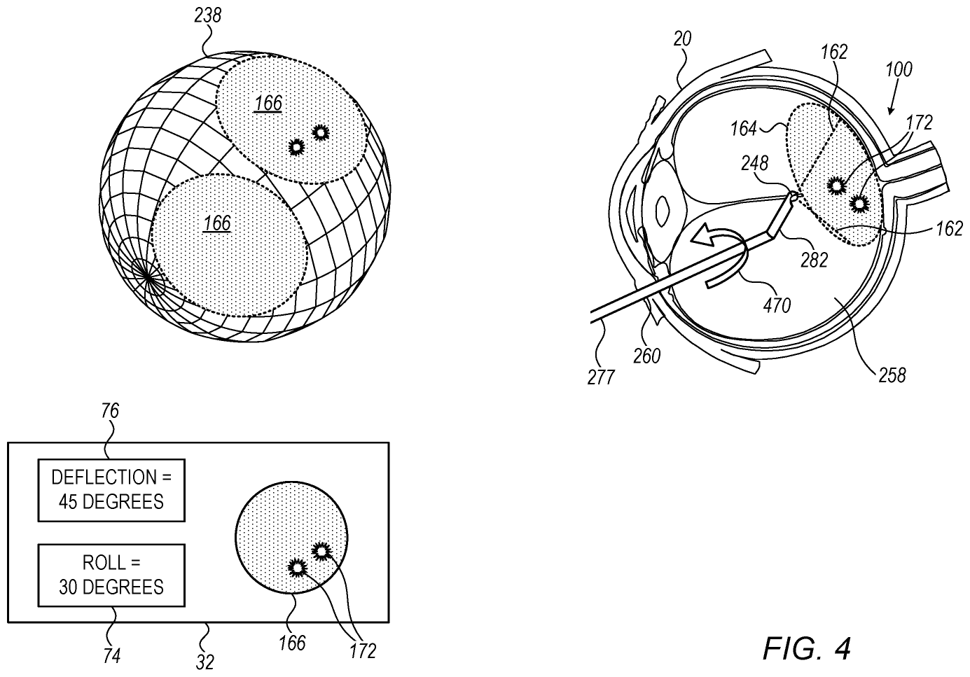

FIG. 4 is a schematic pictorial view of the distal end shaft 277 of the probe of FIG. 2 being used in an examination procedure to inspect vitreous humor 258 in the vicinity of an optical disc 100, in accordance with an embodiment of the present invention. FIG. 4 shows deflectable distal element 282 of shaft 277 of probe 212 inserted into vitreous humor 258 of eye 20 via the incision 260 made during vitrectomy.

The example of FIG. 4 describes deflectable distal element 282 being deflected to 45°, both seen and reported by an indication 76 in display 32. The field-of-view of mirror 248 is shown by way of lines 162 and the image footprint 164. Image 166, captured by the camera, is displayed on display 32 and shows material 172. The example of FIG. 4 assumes that the field-of-view of mirror 248 is about 90°. However, the field-of-view of mirror 248 may be any suitable value less than 90° or greater than 90°. By deflecting deflectable distal element 282 to a positive angle (e.g. +90°) and subsequently to a negative angle (e.g. −90°), and rotating (470) shaft 277 around 360° with each deflection of deflectable distal element 282, the vitreous humor 258 can be captured in full by the camera. The image footprint 164 also moves accordingly to the deflection of deflectable distal element 282, and deflection angles larger than, for example, 120°, allow vitreous humor 258 to be imaged with mirror 248 using a smaller field-of-view to improve image quality.

Display 32 further includes an indication 74 of the current roll of the distal end of deflectable distal element 282 with respect to an original orientation of shaft 277 selected by physician 15 when roll tracking begins. The current roll is computed responsively to signals provided by magnetic orientation sensor 252 (FIG. 2) using any suitable roll computation method. In an embodiment, a further audiovisual indication is given (not shown) that shaft 277 has completed a full rotation.

Material 172, found with probe 212, may subsequently be aspirated from the vitreous humor 258 with a vitrectomy handpiece.

FIG. 4 further shows a schematic view of a three-dimensional surface 238 for use in the apparatus of FIG. 1. The three-dimensional surface 238 may be a sphere or any suitable three-dimensional shape. Images 166 captured by the camera may be coupled on the surface according to the angle of deflection of deflectable distal element 282 and the computed roll angle value of shaft 277 when the respective images were captured by the camera 202 using mirror 248. For example, an origin in a spherical coordinate system may be defined as a suitable point inside the three-dimensional surface 238, for example, at the center of three-dimensional surface 238. The centers of images 166 are then overlaid on the three-dimensional surface 238 according to an azimuthal angle (e.g., the computed roll angle), and a polar angle (e.g., the angle of deflection) of each respective image 166. FIG. 4 shows two images 166 overlaid on the three-dimensional surface 238.

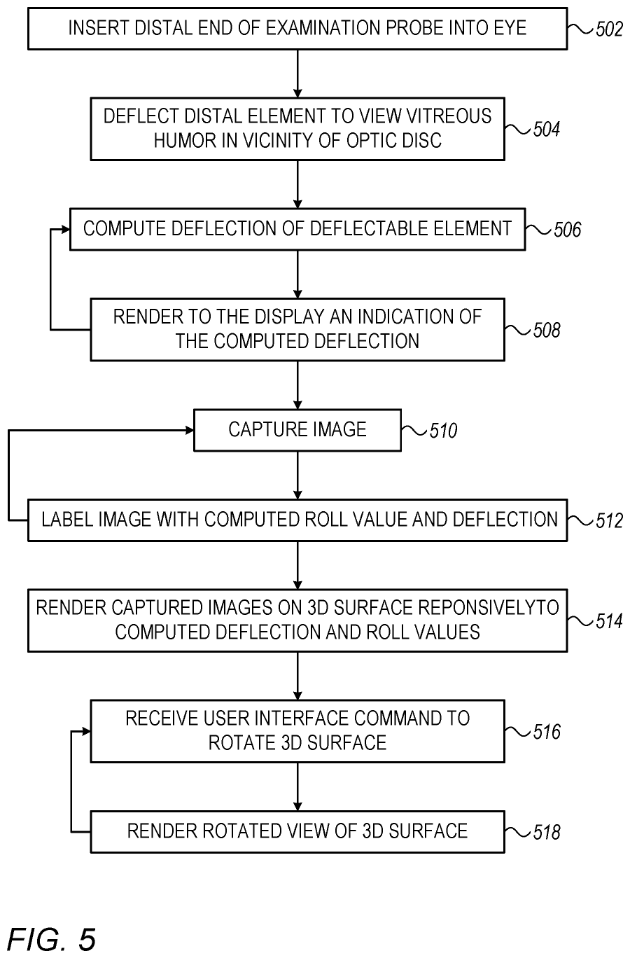

FIG. 5 is a flow chart schematically illustrating a method for using probe 212 of FIG. 2 to examine an eye during or after a vitreoretinal surgery, e.g. vitrectomy, in accordance with an embodiment of the present invention. The process begins with physician 15 inserting a distal end of examination probe 212 into eye 20, at a probe insertion step 502.

Next, physician 15 starts the inspection by deflecting the deflectable distal element 282, e.g., by 90°, to orient mirror 248 posteriorly in order to view, for example, material 172 in the vicinity of optic disc 100, at an inspection alignment step 504.

As the physician deflects deflectable distal element 282, processor 38 computes the deflection of deflectable distal element 282 at a deflection computation step 506, and renders (76) the angle of deflection on display 32, at a deflection angle tracking step 408. Typically steps 506-508 are repeated at intervals to refresh notification 76.

At an image capturing step 510, camera 202 captures an outside volume imaged using mirror 248. The captured image 166 is displayed on display 32.

At a roll angle tracking and deflection step 512, the captured image is labeled with the tracked roll angle (74) of shaft 277 and the deflection angle 76.

Steps 510 and 512 are repeated for multiple images captured at different rolls and, possibly, different deflections of deflectable distal element 282 of probe 212. Processor 32 is configured to render, to display 36 (step 514), the respective images captured by the camera of the respective inside portions of the eye on the three-dimensional surface 238 responsively to the respective computed deflections and the computed roll angle values of the respective images as labeled in step 512.

In a user input receiving step 516, processor 38 receives a user interface command to rotate three-dimensional surface 238. In response, at step 518, processor 38 renders on display 36 a rotated view of the three-dimensional surface 238 responsively to the received user interface command. Steps 516 and 518 may be repeated when a new user interface command is received.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

Various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination.

It will be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. An ophthalmology apparatus, comprising:
a shaft for insertion into an eye;
a convex mirror and a camera coupled at a distal end of the shaft, with the convex mirror located distally to the camera and having an optical path to image onto the camera a region of the eye; and
a magnetic orientation sensor coupled to the shaft and configured to measure a roll angle of the shaft.

2. The apparatus according to claim 1, wherein the region of the eye comprises a capsular bag.

3. The apparatus according to claim 2, further comprising a phacoemulsification needle coupled at the distal end of the shaft.

4. The apparatus according to claim 3, further comprising a pump configured to aspirate a cataract fragment from the capsular bag via the phacoemulsification needle.

5. The apparatus according to claim 1, and comprising a deflectable distal element coupled at the distal end of the shaft, wherein the mirror and the camera are coupled to the deflectable distal element, and wherein the magnetic orientation sensor is coupled to the shaft proximally to the deflectable distal element.

6. The apparatus according to claim 5, wherein the mirror faces away from the deflectable distal element such that the imaged region of a vitreous humor is in a vicinity of an optic disc region while the deflectable distal element is deflected away from the optic disc.

7. The apparatus according to claim 1, further comprising:
a display; and
a processor configured to:
receive respective signals from the magnetic orientation sensor while the shaft is rotated to respective orientations;
compute respective roll angles of the shaft at the respective orientations responsively to the respective received signals;
output a notification responsively to at least one of the computed roll angles; and
render to the display respective images captured by the camera of respective inner regions of the eye.

8. The apparatus according to claim 7, wherein the processor is further configured to:
determine when one of the computed roll angles exceeds a predefined limit; and
output a notification indicating that the one of the computed role angles exceeds the predefined limit.

9. The apparatus according to claim 7, further comprising a deflectable distal element coupled at the distal end of the shaft, wherein the mirror and the camera are coupled to the deflectable distal element, wherein the magnetic orientation sensor is coupled to the shaft proximally to the deflectable distal element, and wherein the processer is further configured to:
compute a deflection of the deflectable distal element; and
render to the display an indication of the computed deflection.

10. The apparatus according to claim 9, further comprising a puller wire passing via a lumen of the shaft, a distal end of the puller wire being connected to the deflectable distal element so that pulling a proximal end of the puller wire deflects the deflectable distal element, wherein the processor is configured to compute the deflection of the deflectable distal element responsively to an electrical impedance of at least part of the puller wire.

11. The apparatus according to claim 9, wherein the processor is configured to:
render to the display the respective images captured by the camera of the respective inner regions of the eye on a three-dimensional surface responsively to the computed deflection and the computed roll angles;
receive a user interface command to rotate the three-dimensional surface; and
render to the display a rotated view of the three-dimensional surface responsively to the received user interface command.

12. An ophthalmology method, comprising:
inserting into an eye a handpiece comprising a shaft, a convex mirror, a camera, and a magnetic orientation sensor, wherein the convex mirror and the camera are coupled at a distal end of the shaft, wherein the convex mirror is located distally to the camera and comprises an optical path to image onto the camera a region of the eye, and wherein the magnetic orientation sensor is coupled to the shaft and configured to measure a roll angle of the shaft;
receiving respective signals from the magnetic orientation sensor while the shaft is rotated to respective orientations;
computing respective roll angles of the shaft at the respective orientations responsively to the respective received signals;
outputting a notification responsively to at least one of the computed roll angles; and
rendering to a display respective images captured by the camera of respective inner regions of the eye.

13. The method according to claim 12, and comprising:
determining when one of the computed roll values exceeds a predefined limit; and
outputting a notification indicating that the one of the computed role values exceeds the predefined limit.

14. The method according to claim 12, and comprising:
deflecting a deflectable distal element that is coupled at the distal end of the shaft, wherein the mirror and the camera are coupled to the deflectable distal element, wherein the magnetic orientation sensor is coupled to the shaft proximally to the deflectable distal element;
computing a deflection of the deflectable distal element; and
rendering to the display an indication of the computed deflection.

15. The method according to claim 14, wherein deflecting the deflectable distal element comprises deflecting the deflectable distal element away from an optic disc of the eye while imaging a region of a vitreous humor in vicinity of the optic disc.

16. The method according to claim 14, wherein computing the deflection includes computing the deflection of the deflectable distal element responsively to an impedance of at least part of a puller wire passing via a lumen of the shaft, a distal end of the puller wire being connected to the deflectable distal element so that pulling a proximal end of the puller wire deflects the deflectable distal element.

17. The method according to claim 14, wherein rendering to the display the indication comprises rendering to the display the respective images captured by the camera of the respective inner regions of the eye on a three-dimensional surface responsively to the computed deflection and the computed roll angles, receiving a user interface command to rotate the three-dimensional surface, and rendering to the display a rotated view of the three-dimensional surface responsively to the received user interface command.

18. The method according to claim 12, wherein the region of the eye comprises a capsular bag.

19. The method according to claim 18, and comprising aspirating a cataract fragment from the capsular bag.
